(19) United States
(12) Patent Application Publication
(10) Pub. No.: US 2013/0073052 A1
(43) Pub. Date: Mar. 21, 2013

(54) STENT WITH IMPROVED END CELL STRUCTURAL MEMBER

(71) Applicants: Seoggwan Kim, West Lafayette, IN (US); Richard A. Swift, South Bend, IN (US)

(72) Inventors: Seoggwan Kim, West Lafayette, IN (US); Richard A. Swift, South Bend, IN (US)

(73) Assignees: Med Institute, West Lafayette, IN (US); Cook Medical Technologies LLC, Bloomington, IN (US)

(21) Appl. No.: 13/579,172

(22) PCT Filed: Feb. 17, 2011
(86) PCT No.: PCT/US11/25184

§ 371 Date: Sep. 18, 2012

Related U.S. Application Data
(60) Provisional application No. 61/305,175, filed on Feb. 17, 2010.

(51) Int. Cl. A61F 2/82

(52) U.S. Cl. CPC A61F 2/82
USPC 623/23.7

(57) ABSTRACT

A stent (30) is provided with an improved structural member (38) at the end (34) of the stent structure (32) to minimize deformation of the stent structure when pushing forces are applied to the end of the stent. The improved structural member is wider than other structural members (40, 42, 44, 46, 48) in the stent structure. The improved structural member is better able to distribute pushing forces to the other structural members in the stent structure with minimal deformation.

### BACKGROUND

The present invention relates generally to medical devices and particularly to a stent with a wide structural member at the proximal or distal end of the stent.

The use of stents to treat various organs, such as the vascular system, colon, biliary tract, urinary tract, esophagus, trachea and the like, has become common in recent years. Stents are most commonly used to treat blockages, occlusions, narrowing ailments and other similar problems that restrict flow through a passageway. One area where stents are commonly used for treatment involves implanting an endovascular stent into the vascular system in order to improve or maintain blood flow through narrowed arteries. Stents have been shown to be useful in treating various vessels throughout the vascular system, including both coronary vessels and peripheral vessels (e.g., carotid, brachial, renal, iliac and femoral).

The use of stents in coronary vessels has drawn particular attention from the medical community because of the growing number of people each year that suffer from heart problems associated with stenosis (i.e., narrowing of a vessel). This has led to an increased demand for medical procedures to treat such problems. The widespread frequency of heart problems may be due to a number of societal changes, including the tendency of people to exercise less and the prevalence of unhealthy diets, in conjunction with the fact that people generally have longer life spans now than previous generations. Stents have become a popular alternative for treating coronary stenosis because stenting procedures are considerably less invasive than conventional procedures. Traditionally, stenosis of the coronary arteries has been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient. Vascular stents are also commonly used to treat many different peripheral arteries due to the minimally invasive nature of stenting procedures. To address the growing demand for minimally invasive medical procedures for the treatment of coronary arteries, peripheral arteries and other passageway problems, the medical community has begun to turn away from conventional invasive procedures like bypass surgery and increasingly the treatment of choice now involves a variety of stenting procedures.

Many different types of stents and stenting procedures are possible. In general, however, stents are typically designed as tubular support structures that may be inserted percutaneously and transluminally through a body passageway. Traditionally, stents are made from a metal or other synthetic material with a series of radial openings extending through the support structure of the stent to facilitate compression and expansion of the stent. Although stents may be made from many types of materials, including non-metallic materials, common examples of metallic materials that may be used to make stents include stainless steel, nitinol, cobalt-chrome alloys, amorphous metals, tantalum, platinum, gold and titanium. Typically, stents are implanted within a passageway by positioning the stent within the area to be treated and then expanding the stent from a compressed diameter to an expanded diameter. The ability of the stent to expand from a compressed diameter makes it possible to thread the stent to the area to be treated through various narrow body passageways while the stent is in the compressed diameter. Once the stent has been positioned and expanded at the area to be treated, the tubular support structure of the stent contacts and radially supports the inner wall of the passageway. As a result, the implanted stent mechanically prevents the passageway from narrowing and keeps the passageway open to facilitate fluid flow through the passageway.

Stents can generally be characterized as either balloon-expandable or self-expanding. However, stent designs and implantation procedures vary widely. For example, although physicians often prefer particular types of stents for certain types of procedures, the uses for balloon-expandable and self-expanding stents frequently overlap and procedures related to one type of stent are frequently adapted to other types of stents.

Balloon-expandable stents are generally used to treat stenosis of the coronary arteries. Usually, balloon-expandable stents are made from ductile materials that plastically deform relatively easily. In the case of stents made from metal, 316L stainless steel that has been annealed is a common choice for this type of stent. One procedure for implanting balloon-expandable stents involves mounting the stent circumferentially on the balloon of a balloon-tipped catheter and threading the catheter through a vessel passageway to the area to be treated. Once the balloon is positioned at the narrowed portion of the vessel to be treated, the balloon is expanded by pumping saline through the catheter to the balloon. As a result, the balloon simultaneously dilates the vessel and radially expands the stent within the dilated portion. The balloon is then deflated and the balloon-tipped catheter is retracted from the passageway. This leaves the expanded stent permanently implanted at the desired location. Ductile metal lends itself to this type of stent since the stent may be compressed by plastic deformation to a small diameter when mounted onto the balloon. When the balloon is later expanded in the vessel, the stent is once again plastically deformed to a larger diameter to provide the desired radial support structure. Traditionally, balloon-expandable stents have been more commonly used in coronary vessels than in peripheral vessels because of the deformable nature of these stents. One reason for this is that peripheral vessels tend to experience frequent traumas from external sources (e.g., impacts to a person's arms, legs, etc.) which are transmitted through the body's tissues to the vessel. In the case of peripheral vessels, there is an increased risk that an external trauma could cause a balloon-expandable stent to once again plastically deform in unexpected ways with potentially severe and/or catastrophic results. In the case of coronary vessels, however, this risk is minimal since coronary vessels rarely experience traumas transmitted from external sources.

Self-expanding stents are increasingly used and accepted by physicians for treating a variety of ailments. Self-expanding stents are usually made of shape memory materials or materials that act like a spring. Typical metals used in this type of stent include nitinol and 304 stainless steel. A common procedure for implanting a self-expanding stent involves a two-step process. First, the narrowed vessel portion to be treated is dilated with a balloon but without a stent mounted on the balloon. Second, a stent is implanted into the dilated vessel portion. To facilitate stent implantation, the stent is installed on the end of a catheter in a compressed, small diameter state and is usually retained in the small diameter by inserting the stent into a sheath at the end of the catheter. The stent is then guided to the balloon-dilated portion and is released from the catheter by pulling the retaining sheath off the stent. Once released from the retaining sheath, the stent radially springs outward to an expanded diameter until the stent contacts and presses against the vessel wall. Traditionally, self-expanding stents have been more commonly used in peripheral vessels than in coronary vessels due to the shape memory characteristic of the metals that are used in these stents. One advantage of self-expanding stents for peripheral vessels is that traumas from external sources do not permanently deform the stent. Instead, the stent may temporarily deform during an unusually harsh trauma but will spring back to its expanded state once the trauma is relieved. Self-expanding stents, however, are often considered to be less preferred for coronary vessels as compared to balloon-expandable stents. One reason for this is that balloon-expandable stents can be precisely sized to a particular vessel diameter and shape since the ductile metal that is used can be plastically deformed to a desired size and shape. In contrast, self-expanding stents are designed with a particular expansible range. Thus, after being implanted, self-expanding stents continue to exert pressure against the vessel wall.

Typically, stents are provided with markers and/or pushing members that are attached to the proximal and/or distal ends of the stent structure. These features may be used for a number of purposes and usually serve more than one function. For example, markers are usually provided at both the proximal and distal ends of the stent to assist the physician in positioning the stent during stenting procedures. Generally, separate markers are needed on most stents since the stent structure itself cannot usually be seen easily on x-ray and other visualization equipment. This is due in part to the types of material that are usually used in stent structures and the slenderness of the structural members in the stent structure. Markers address this visualization problem by providing features with increased radiopacity along the proximal and distal ends of the stent. The features (i.e., the markers) are typically larger in width than the structural members of the stent structure and are usually filled with a radiopaque material like gold or platinum. As a result, the radiopaque material in the markers can be seen more easily on the physician's visualization equipment than the stent structure itself.

Pushing members are also used at the proximal and/or distal ends of many stents. Pushing members are particularly useful for self-expanding stents but may also be used on balloon-expandable stents. In either case, the pushing members provide a separate contact surface at the end of the stent that may be pushed against. As a result, the stent structure itself is not directly pushed against. In the case of self-expanding stents, the pushing members of the stent are used at several different times. For example, during the manufacture of self-expanding stents and their corresponding delivery systems, the stent must be loaded into the delivery system in a compressed state. Delivery systems for self-expanding stents are well known to those in the art, and therefore, a detailed description is not necessary. However, as described above, delivery systems for self-expanding stents usually include a retaining sheath at the end of a catheter which restrains the outer surface of the stent and keeps the stent compressed until the stent is released at the site of implantation. A common manufacturing method for loading stents into the retaining sheath involves compressing the stent while at the same time pushing on one end of the stent in order to slide the stent into the sheath. Typical machines for compressing self-expanding stents include iris compressors and thin Mylar sheets that are wrapped around the stent and pulled tight to compress the stent within the sheet. The stent may be pushed out of these machines by using a solid or tubular rod to push on one end of the stent to force the stent out the opposite side of the machine. The stent may also be compressed and pushed into a transfer tube first and then pushed again later through the transfer tube into the delivery system.

Pushing members are also used on the proximal end of self-expanding stents in order to release the stent from the delivery system for implantation. As previously described, self-expanding stents are released for implantation by pulling the retaining sheath off the stent. Typically, the delivery system also includes a holder within the retaining sheath which contacts the proximal end of the stent. Generally, the holder and the sheath are designed to move relative to each other so that as the sheath is pulled back, the holder can be maintained in place. As a result, the holder prevents the stent from moving rearward with the retaining sheath as the sheath is pulled back. In effect, the stent is pushed out of the sheath by the holder as the sheath is pulled rearward.

Typically, the markers on a stent are also used as pushing members and vice versa. One problem with current stent structures is that the pushing force that is transmitted by the pushing member to the stent structure is concentrated onto the structural members of the stent that are directly connected to the pushing member. As a result, the pushing force can cause the structural members to bend and deform as the stent is being pushed. In extreme cases, this concentrated force can permanently deform parts of the stent structure. This problem may be of particular concern on longer length stents. Generally, most stents that are currently used for medical treatments are 8 cm or less in length. However, stents that are longer than 8 cm may become more common to treat various peripheral arteries, such as the superficial femoral artery. When longer stents like these are pushed, either during loading into the delivery system or during release, higher frictional forces must be overcome in order to move the stent. The longer length of some of these stents also makes the stents generally less stable than shorter stents. As a result, the bending and deforming problems that may occur when pushing on a stent tend to be more pronounced and damaging on longer stents. Deformation caused by pushing forces may also be a particular problem with small diameter stents used in the smaller peripheral arteries. The size of stents typically used to treat these types of vessels is usually 4 or 5 French in diameter. Smaller peripheral arteries generally exist in the legs, arms and neck regions. Because of the location of these arteries, they typically experience higher degrees of movement and shape change when a person engages in daily activities. Thus, a stent implanted in these regions typically requires a higher degree of axial, radial and torsional flexibility. In addition, it is desirable for such stents to provide high radial strength to maintain vessel patency. However, this combination of high flexibility and high radial strength may make these types of stents more susceptible to deformation caused by pushing forces. Although these types of problems relate particularly to self-expanding stents, other types of stents, such as balloon-expandable stents, may experience similar types of problems.

Deformation of the stent structure is undesirable for a number of reasons. Foremost, it is difficult or impossible in many situations to identify deformation before a stent has been implanted into a patient's body. For example, during manufacturing, retaining sheaths and transfer tubes are often opaque or difficult to see through, which makes visual inspections unreliable. In addition, some undesirable deformations may be so small dimensionally that they are virtually undistinguishable. Deformations may also occur at either end of the stent, especially when a transfer tube is used during loading and opposite ends of the stent are pushed at different stages of the loading process. Further, when deformation occurs during deployment of a stent by a physician within a patient's body, there is no practical way to inspect the stent during deployment and retrieve a deformed stent.

Structural deformations of a stent may have a number of undesirable results. One concern is that the fatigue life of a stent may be reduced due to deformation. Fatigue life may be reduced directly at the point where the deformation occurred or may be reduced elsewhere in the stent structure. For example, when a portion of the distal or proximal end of a stent has been permanently deformed, this condition can cause a non-uniform propagation of the deformation along a portion of the length of the stent. In other words, when one side of a stent has been deformed at an end of the stent, that side of the stent may stretch to accommodate the deformation relative to the opposite side that has not been deformed. This can cause strain concentrations at other locations in the stent structure that are not immediately near the original point of deformation. In addition to reduced fatigue life, deformations of the stent structure can create other concerns as well if the deformation causes part of the stent structure to protrude partially into the vessel lumen or against the vessel wall. For example, if part of the stent structure significantly protrudes into the lumen of the vessel, the protrusion may interfere with blood flow and could become an initiation site for emboli formation. On the other hand, if part of the stent structure significantly protrudes against the vessel wall, scarring of the vessel tissues could occur.

Accordingly, the inventor believes it would be desirable to provide an improved stent structure. A solution to these and other problems is described more fully below.

### SUMMARY

A stent structure is described that minimizes deformation of the stent structure when pushing forces are applied to the end of the stent. The stent structure includes an improved structural member that is wider than other structural members in the stent structure. The improved structural member distributes the pushing forces to the other structural members in the stent structure with minimal deformation.

The invention may include any of the following aspects in various combinations and may also include any other aspect described below in the written description or in the attached drawings.

A stent, comprising
a series of structural members extending around a circumference from a first end to a second end, the structural members being expandable between a collapsed diameter and an expanded diameter;
wherein the structural members forming one of the first and second ends comprise a series of first structural members and a series second structural members, wherein each of the first structural members are circumferentially interconnected by the second structural members;
a pushing member attached to an outer end of each of the first structural members; and
wherein said first structural members are at least 1.5 times as wide as said second structural members.

The stent wherein the stent is about 5 French or less in diameter.

The stent according to claim 1, wherein the stent is between about 4 and about 5 French in diameter.

The stent wherein the first structural members are at least 2.25 times wider than the second structural members.

The stent wherein the stent is self-expanding.

The stent wherein a cross-section of the series of structural members is generally rectangular.

The stent wherein the pushing member is a round eyelet.

The stent wherein each of the pushing members is attached to one of the second structural members on both sides of each of the first structural members.

The stent wherein each of the first structural members is circumferentially interconnected to a circumferentially adjacent first structural member by six of the second structural members oriented in a sinusoidal pattern.

The stent wherein the first structural members comprise three structural members.

The stent wherein the series of structural members further comprise a series of third structural members, the third structural members being oriented in a sinusoidal ring around the circumference, wherein an inner end of each of the first structural members is attached to two of the third structural members, the first structural members being approximately as long as the second structural members and being longitudinally offset from the third structural me

The stent wherein the third structural members oriented in the sinusoidal ring comprise 18 structural members.

The stent wherein the series of structural members further comprise a series of fourth structural members, a series of fifth structural members and a series of sixth structural members oriented in a sinusoidal ring around the circumference, wherein one end of each of the fourth structural members is attached to two of the third structural members and another end of the fourth structural members is attached to two of the sixth structural members, the fourth structural members being approximately as long as the sixth structural members and being circumferentially adjacent the sixth structural members, wherein one end of each of the fifth structural members is attached to two of the sixth structural members and another end of the fifth structural members extends away from the third structural members, the fifth structural members being approximately as long as the sixth structural members and being circumferentially adjacent the sixth structural members, wherein the sixth structural members are oriented in a sinusoidal pattern and circumferentially interconnect the fourth and fifth structural members.

The stent wherein the fourth structural members comprise three structural members, the fifth structural members comprise three structural members, and the sixth structural members comprise 18 structural members.

The stent wherein a width of the fourth, fifth and sixth structural members is substantially equal to the width of a second structural members.

A stent, comprising:
a series of structural members extending around a circumference from a first end to a second end, the structural members being self-expanding from a collapsed diameter to an expanded diameter, wherein a cross-section of the structural members is generally rectangular;
wherein the structural members forming one of the first and second ends comprise a series of first structural members and a series second structural members, wherein each of the first structural members are circumferentially interconnected by the second structural members;
a pushing member attached to an outer end of each of the first structural members, wherein each of the pushing members is attached to one of the second structural members on both sides of each of the first structural members;
wherein the first structural members are at least 2.25 times wider than the second structural members;
wherein the series of structural members further comprise a series of fourth structural members, a series of fifth structural members and a series of sixth structural members oriented in a sinusoidal ring around the circumference, wherein one end of each of the fourth structural members is attached to the first structural members through a series of third structural members and another end of the fourth structural members is attached to two of the sixth structural members, the fourth structural members being approximately as long as the sixth structural members and being circumferentially adjacent the sixth structural members, wherein one end of each of the fifth structural members is attached to two of the sixth structural members and another end of the fifth structural members extends away from the third structural members, the fifth structural members being approximately as long as the sixth structural members and being circumferentially adjacent the sixth structural members, wherein the sixth structural members are oriented in a sinusoidal pattern and circumferentially interconnect the fourth and fifth structural members; and wherein a width of the fourth, fifth and sixth structural members is substantially equal to a width of the second structural members.

The stent wherein each of the first structural members is circumferentially interconnected to a circumferentially adjacent first structural member by six of the second structural members oriented in a sinusoidal pattern, the first structural members comprising three structural members, the fourth structural members comprising three structural members, the fifth structural members comprising three structural members, and the sixth structural members comprising 18 structural members.

The stent wherein the stent is between about 4 and about 5 French in diameter.

The stent wherein the third structural members are oriented in a sinusoidal ring around the circumference, wherein an inner end of each of the first structural members is attached to two of the third structural members and the one end of the fourth structural members is attached to two of the third structural members, the first structural members being approximately as long as the second structural members and being longitudinally offset from the third structural members.

The stent wherein the third structural members oriented in the sinusoidal ring comprise 18 structural members.

### BRIEF DESCRIPTION OF SEVERAL VIEWS OF THE DRAWINGS

The invention may be more fully understood by reading the following description in conjunction with the drawings, in which:

FIG. 1A is a flat plan view of an unexpanded conventional stent structure;

FIG. 1B is a side perspective view of an expanded conventional stent structure;

FIG. 1C is an enlarged plan view of a portion of an expanded conventional stent structure;

FIG. 2A is a flat plan view of an unexpanded improved stent structure; and

FIG. 2B is an enlarged plan view of an expanded improved stent structure.

### DETAILED DESCRIPTION

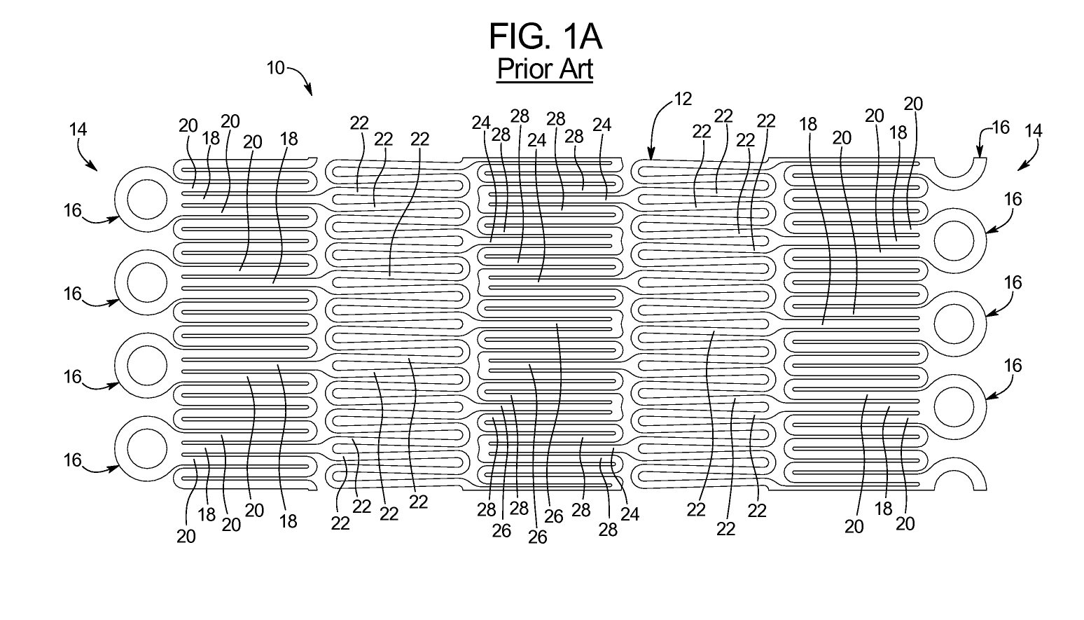

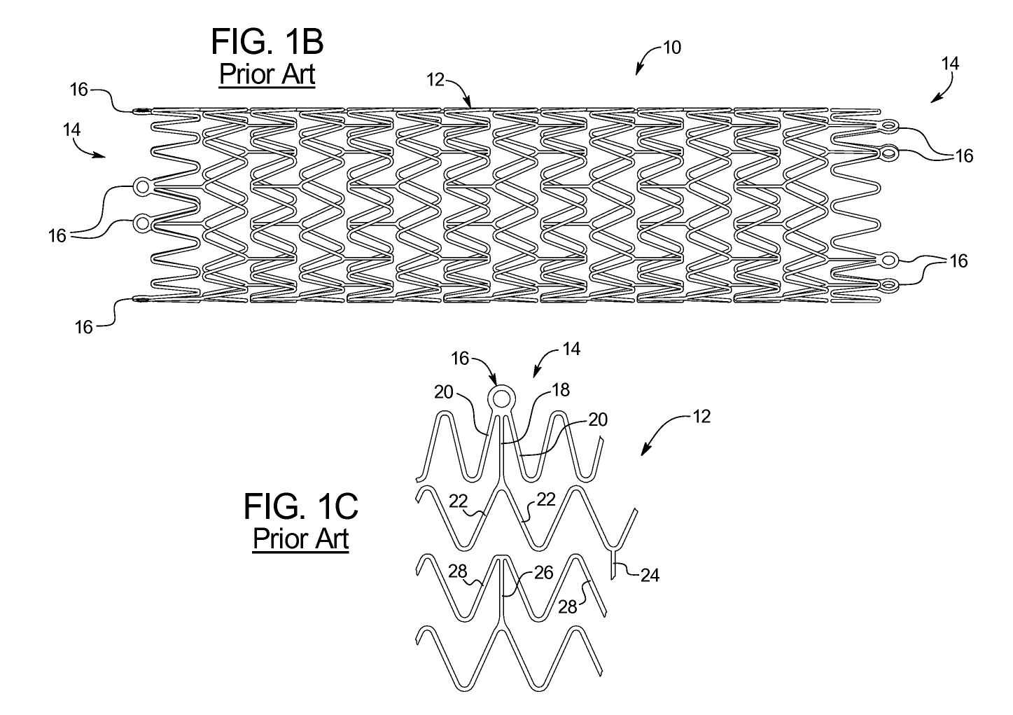

Referring now to the figures, and particularly to FIGS. 1A-1C, a conventional self-expanding stent structure 12 is shown. Although the figures herein are shown as flat views of the disclosed stents in laid-out patterns, it is understood that the stent structures are preferably cylindrical in shape and the views shown in the figures may be wrapped around the cylindrical shape of the stent. The stent structure 12 is formed from a series of interconnected structural members 18, 20, 22, 24, 26, 28. Although stent structures 12 may be made from various methods and may have various shapes, the structures 12, 32 described herein may be cut from a cannula using a laser. Thus, the cross-sectional shape of the structural members 18, 20, 22, 24, 26, 28 is generally rectangular. At the end 14 of the stent 10, which may be the distal end, proximal end or both ends, a series of markers 16 may be provided. The markers 16 may be formed as eyelets 16 with an opening through each eyelet 16 so that a radiopaque material may be inserted through the openings. The eyelets 16 may be used as pushing members 16 to push on the ends 14 of the stent 10 to apply axial force to the stent structure 12. Although the pushing members 16 are shown as marker eyelets 16, it is understood that the pushing members 16 may take other forms as well.

The pushing members 16 are each attached to a first structural member 18 and two second structural members 20 that are disposed on each side of the first structural members 18. The second structural members 20 are interconnected through a series of second structural members 20 to form a sinusoidal pattern that circumferentially interconnects the first structural members 18. Thus, the second structural members 20 form a sinusoidal ring around the cylindrical stent structure 12. The first structural members 18 are generally about as long as the second structural members 20 and are circumferentially adjacent the second structural members 20. The inner end of each first structural member 18 is attached to third structural members 22. The third structural members 22 form a sinusoidal ring that wraps around the cylindrical stent structure 12. Preferably, as shown, the third structural members 22 angle toward each other as they extend away from each bend that interconnects the third structural members 22. A series of fourth structural members 24 are attached to the third structural members 22 on the opposite side of the first structural members 18. One end of each fourth structural member 24 is attached to two of the third structural members 22. The other end of each of the fourth structural members 24 is attached to two of the sixth structural members 28. The fourth structural members 24 are generally about as long as the sixth structural members 28 and are circumferentially adjacent to the sixth structural members 28. The sixth structural members 28 are interconnected to each other to form a sinusoidal pattern around the cylindrical stent structure 12. A series of fifth structural members 26 are also attached to the sixth structural members 28. One end of each of the fifth structural members 26 is attached to two of the sixth structural members 28. The other ends of the fifth structural members 26 extend away from the third structural members 22 and are attached to additional structural members in the stent structure 12. Accordingly, the pattern of third, fourth, fifth and sixth structural members 22, 24, 26, 28 may be repeated along the length of the stent 10 as desired. Preferably, the opposing end of the stent structure 12 has an end region like that described above with pushing members 16 and first and second structural members 18, 20.

Typically, in the stent structure 12 described above, the width of the first, second, third, fourth, fifth and sixth structural members 18, 20, 22, 24, 26, 28 are all equal. This is desirable for a number of reasons. For example, equal width structural members throughout the stent structure 12 makes the stent structure 12 simpler to design and analyze. In addition, equal width structural members throughout also makes manufacturing and quality control of the stent structure 12 easier. The performance of the stent structure 12 once it has been implanted in a patient's body is also easier to predict with equal width structural members throughout the stent structure 12. However, one problem that may be experienced with the described stent structure 12 is that the first structural members 18 may be susceptible to the deforming and buckling when force is applied to the pushing members 16 during loading of the stent 10 into a delivery system or deploying the stent 10 from the delivery system. This is undesirable for a number of reasons as described further above.

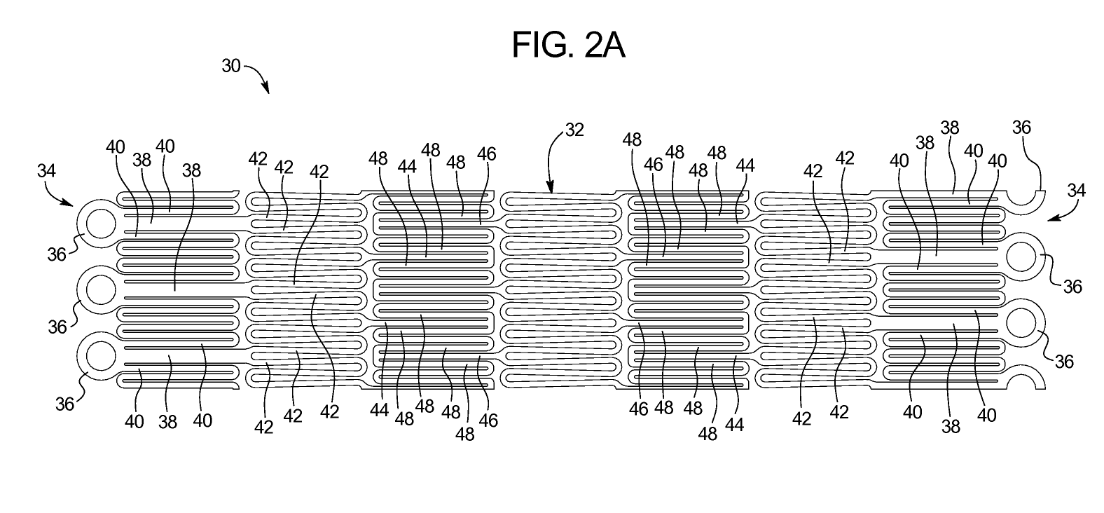

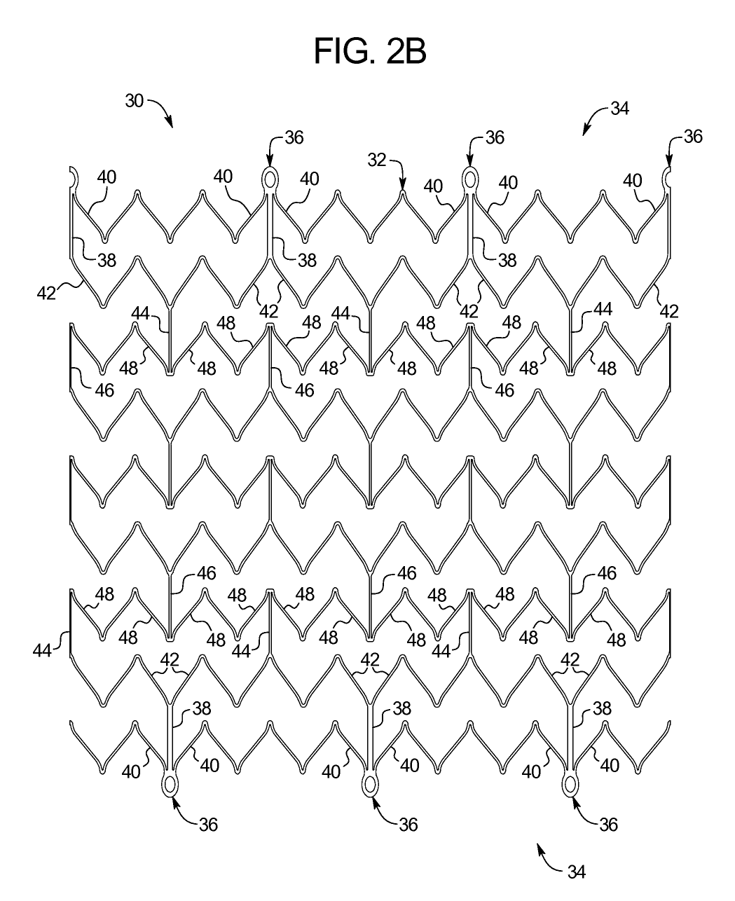

Turning now to FIGS. 2A-2B, an improved stent structure 32 for a stent 30 is shown. The stent structure 32 includes pushing members 36, first structural members 38, second structural members 40, third structural members 42, fourth structural members 44, fifth structural members 46 and sixth structural members 48 in a similar pattern as described above. Although the general pattern of the improved stent structure 32 is similar to the stent structure 12 described above, the dimensions, relative sizes, number of structural members and other features may be different from the stent structure 12 described above. The stent structure 32 is particularly useful for self-expanding stents 30 in the 4 to 5 French size diameter range or 5 French or smaller. This type of stent 30 may be used in extreme peripheral arteries that require high axial, radial and torsional flexibility and high radial strength. In particular, the stent structure 32 preferably includes three pushing members 36 and three first structural members 38 at the end 34 of the stent structure 32. Between each of the first structural members 38, six second structural members 40 may be provided. Thus, the end 34 of the stent structure 32 is provided with a total of 18 second structural members 40. Preferably, both ends of the stent 30 are provided with the end 34 shown in FIG. 2, but the opposite end of the stent 30 may be provided with a different structure if desired. Preferably, the stent structure 32 is also provided with 18 third structural members 42 and 18 sixth structural members 48. Further, the stent structure 32 is preferably provided with three fourth structural members 44 and three fifth structural members 46. The patterns of the third, fourth, fifth and sixth structural members 42, 44, 46, 48 may be repeated along the length of the stent 30 to provide a stent structure 32 with the overall length that is desired.

In the improved stent structure 32, the width of each of the first structural members 38 is wider than the second structural members 40 and may also be wider than the third, fourth, fifth and sixth structural members 42, 44, 46, 48. Preferably, the width of the second, third, fourth, fifth and sixth structural members 40, 42, 44, 46, 48 are all equal. For example, the width of the second, third, fourth, fifth and sixth structural members 40, 42, 44, 46, 48 may be about 0.13 mm, while the width of the first structural members 38 may be about 0.3 mm. However, it is preferable for the width of the first structural members 38 to be at least 1.5 times as wide as the second structural members 40. However, it may be more preferable for the width of the first structural members 38 to be at least twice as wide or at least 2.25 times as wide as the second structural members 40. Similar ratios between the width of the first structural members 38 and the width of the third, fourth, fifth and sixth structural members 42, 44, 46, 48 are also preferred.

The improved stent structure 32 is particularly useful for minimizing deformation of the stent structure 32 when the end 34 of the stent 30 is pushed during loading of the stent 30 into a delivery system and deployment of the stent 30 in a patient's body. Because the first structural members 38 are wider than conventional first structural members 18 and wider than the other structural members 40, 42, 44, 46, 48 in the stent structure 32, the first structural members 38 are able to transfer pushing forces to the other structural members 40, 42, 44, 46, 48 in the stent structure 32 with minimal deformation of the first structural members 38. In addition, by arranging the first, second, third, fourth, fifth and sixth structural members 38, 40, 42, 44, 46, 48 in the pattern described above, the improved stent structure 32 is able to be compressed to an equivalent compressed diameter of a conventional stent. Thus, the improved stent structure 32 is able to withstand pushing forces better than conventional stent structures 12, while the overall compressed and expanded diameters of the improved stent structure 32 can be the same as conventional stent structures. Moreover, the improved stent structure 32 may be particularly beneficial for stents 30 used in extreme peripheral arteries, since these stents 30 are often long, small in diameter and very flexible with high radial strength. This combination of factors may make these types of stents 30 more prone to deformation problems in the region of the first structural members 38 than other conventional stents that are used in other applications and have different sizes.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. A stent, comprising
a series of structural members extending around a circumference from a first end to a second end, said structural members being expandable between a collapsed diameter and an expanded diameter;
wherein said structural members forming one of said first and second ends comprise a series of first structural members and a series second structural members, wherein each of said first structural members are circumferentially interconnected by said second structural members;
a pushing member attached to an outer end of each of said first structural members; and
wherein said first structural members are at least 1.5 times as wide as said second structural members.

2. The stent according to claim 1, wherein said stent is about 5 French or less in diameter.

3. The stent according to claim 1, wherein said stent is between about 4 and about 5 French in diameter.

4. The stent according to claim 1, wherein said first structural members are at least 2.25 times wider than said second structural members.

5. The stent according to claim 1, wherein said stent is self-expanding.

6. The stent according to claim 1, wherein a cross-section of said series of structural members is generally rectangular.

7. The stent according to claim 1, wherein said pushing member is a round eyelet.

8. The stent according to claim 1, wherein each of said pushing members is attached to one of said second structural members on both sides of each of said first structural members.

9. The stent according to claim 8, wherein each of said first structural members is circumferentially interconnected to a circumferentially adjacent first structural member by six of said second structural members oriented in a sinusoidal pattern.

10. The stent according to claim 9, wherein said first structural members comprise three structural members.

11. The stent according to claim 10, wherein said series of structural members further comprise a series of third structural members, said third structural members being oriented in a sinusoidal ring around said circumference, wherein an inner end of each of said first structural members is attached to two of said third structural members, said first structural members being approximately as long as said second structural members and being longitudinally offset from said third structural members.

12. The stent according to claim 11, wherein said third structural members oriented in said sinusoidal ring comprise 18 structural members.

13. The stent according to claim 12, wherein said series of structural members further comprise a series of fourth structural members, a series of fifth structural members and a series of sixth structural members oriented in a sinusoidal ring around said circumference, wherein one end of each of said fourth structural members is attached to two of said third structural members and another end of said fourth structural members is attached to two of said sixth structural members, said fourth structural members being approximately as long as said sixth structural members and being circumferentially adjacent said sixth structural members, wherein one end of each of said fifth structural members is attached to two of said sixth structural members and another end of said fifth structural members extends away from said third structural members, said fifth structural members being approximately as long as said sixth structural members and being circumferentially adjacent said sixth structural members, wherein said sixth structural members are oriented in a sinusoidal pattern and circumferentially interconnect said fourth and fifth structural members.

14. The stent according to claim 13, wherein said fourth structural members comprise three structural members, said fifth structural members comprise three structural members, and said sixth structural members comprise 18 structural members.

15. The stent according to claim 14, wherein a width of said fourth, fifth and sixth structural members is substantially equal to said width of a second structural members.

16. A stent, comprising:
a series of structural members extending around a circumference from a first end to a second end, said structural members being self-expanding from a collapsed diameter to an expanded diameter, wherein a cross-section of said structural members is generally rectangular;
wherein said structural members forming one of said first and second ends comprise a series of first structural members and a series second structural members, wherein each of said first structural members are circumferentially interconnected by said second structural members;
a pushing member attached to an outer end of each of said first structural members, wherein each of said pushing members is attached to one of said second structural members on both sides of each of said first structural members;
wherein said first structural members are at least 2.25 times wider than said second structural members;
wherein said series of structural members further comprise a series of fourth structural members, a series of fifth structural members and a series of sixth structural members oriented in a sinusoidal ring around said circumference, wherein one end of each of said fourth structural members is attached to said first structural members through a series of third structural members and another end of said fourth structural members is attached to two of said sixth structural members, said fourth structural members being approximately as long as said sixth structural members and being circumferentially adjacent said sixth structural members, wherein one end of each of said fifth structural members is attached to two of said sixth structural members and another end of said fifth structural members extends away from said third structural members, said fifth structural members being approximately as long as said sixth structural members and being circumferentially adjacent said sixth structural members, wherein said sixth structural members are oriented in a sinusoidal pattern and circumferentially interconnect said fourth and fifth structural members; and
wherein a width of said fourth, fifth and sixth structural members is substantially equal to a width of said second structural members.

17. The stent according to claim 16, wherein each of said first structural members is circumferentially interconnected to a circumferentially adjacent first structural member by six of said second structural members oriented in a sinusoidal pattern, said first structural members comprising three structural members, said fourth structural members comprising three structural members, said fifth structural members comprising three structural members, and said sixth structural members comprising 18 structural members.

18. The stent according to claim 17, wherein said stent is between about 4 and about 5 French in diameter.

19. The stent according to claim 18, wherein said third structural members are oriented in a sinusoidal ring around said circumference, wherein an inner end of each of said first structural members is attached to two of said third structural members and said one end of said fourth structural members is attached to two of said third structural members, said first structural members being approximately as long as said second structural members and being longitudinally offset from said third structural members.

20. The stent according to claim 19, wherein said third structural members oriented in said sinusoidal ring comprise 18 structural members.
